(19) United States
(12) Patent Application Publication
(10) Pub. No.: US 2017/0227512 A1
(43) Pub. Date: Aug. 10, 2017

(54) METHOD FOR DEMONSTRATING STAIN REMOVAL

(71) Applicant: COLGATE-PALMOLIVE COMPANY, New York, NY (US)

(72) Inventors: Neelima UTGIKAR, Maharashtra (IN); Maya BHANSALI, Mumbai (IN); Ekta SHAH, Maharashtra (IN); Shashank POTNIS, Thane (W) (IN); Richard SULLIVAN, Atlantic Highlands, NJ (US)

(73) Assignee: COLGATE-PALMOLIVE COMPANY, New York, NY (US)

(21) Appl. No.: 15/319,608

(22) PCT Filed: Jun. 20, 2014
(86) PCT No.: PCT/US2014/043400

§ 371 Date: Dec. 16, 2016

(51) Int. Cl. G01N 33/15; G09B 19/00; A61K 8/25; A61K 8/24; G01N 21/88; A61Q 11/00

(52) U.S. Cl. CPC G01N 33/15; G01N 21/8803; A61Q 11/00; A61K 8/25; A61K 8/24; G09B 19/0084

(57) ABSTRACT

An in vitro method for demonstrating the stain removal efficacy of an oral care composition comprising (a) providing a substrate, (b) providing a staining solution, (c) providing a treatment solution comprising the oral care composition, (d) immersing the substrate in the staining solution, (e) removing the substrate from the staining solution, (f) evaluating the color of the stained substrate, (g) applying the treatment solution to the substrate and (h) evaluating the color of the treated substrate, wherein the staining solution comprises coffee is provided.

The present invention relates to methods for demonstrating stain removal. In particular, the present invention relates to in vitro methods for demonstrating stain removal by oral care compositions. The present invention also relates to staining solutions and their use in methods for demonstrating stain removal.

### BACKGROUND

White teeth are seen as desirable by consumers and a variety of oral care compositions with the ability to remove stains from teeth are available. Oral care compositions with whitening efficacy can remove extrinsic stains and thereby whiten teeth. However, the stain removal process is gradual and it may take time for a consumer to notice the benefit. In certain circumstances it may take a week of brushing twice a day for a detectable shade difference to develop.

It would be desirable to be able to demonstrate the stain removal benefit of an oral care composition instantly to consumers.

### BRIEF SUMMARY

According to one aspect of the present invention there is provide an in vitro method for demonstrating the stain removal efficacy of an oral care composition comprising
a. providing a substrate,
b. providing a staining solution,
c. providing a treatment solution comprising the oral care composition,
d. immersing the substrate in the staining solution,
e. removing the substrate from the staining solution,
f. evaluating the color of the stained substrate,
g. applying the treatment solution to the substrate and
h. evaluating the color of the treated substrate,
   wherein the staining solution comprises coffee.

Optionally the staining solution comprises instant coffee powder. Optionally the staining solution comprises instant coffee powder and an acid. Optionally the staining solution comprises instant coffee powder at a concentration of from about 0.10 g/ml to about 0.60 g/ml. Optionally the staining solution comprises instant coffee powder at a concentration of from about 0.20 g/ml to about 0.40 g/ml. Optionally the staining solution comprises instant coffee powder at a concentration of about 0.25 g/ml.

Optionally the staining solution comprises acetic acid. Optionally the staining solution comprises from about 0.5% to about 2.0% by volume glacial acetic acid. Optionally the staining solution comprises about 1.25% by volume glacial acetic acid. Optionally the staining solution comprises instant coffee powder at a concentration of about 0.25 g/ml and glacial acetic acid at a concentration of about 1.25% by volume.

Optionally the substrate comprises calcium carbonate. Optionally the substrate comprises the exoskeleton of a marine bivalve mollusk. Optionally the substrate comprises the exoskeleton of a marine bivalve mollusk in the family Arcidae.

Optionally the oral care composition is a dentifrice. Optionally the oral care composition comprises silica and pyrophosphate. Optionally the treatment solution comprises a slurry of an oral care composition and water. Optionally the treatment solution comprises a slurry of a dentifrice and water in a ratio of from about 1:2 to about 1:4 by weight. Optionally the treatment solution comprises a slurry of a dentifrice and water in a ratio of about 1:3 by weight.

Optionally step d comprises immersing the substrate in the staining solution for from about 20 to about 60 minutes. Optionally step d comprises immersing the substrate in the staining solution for about 45 minutes. Optionally the method additionally comprises step e (ii) wherein the substrate is air-dried.

Optionally step d comprises immersing the substrate in the treatment solution for a period of from about 20 seconds to about 2 minutes. Optionally step g comprises immersing the substrate in the treatment solution for a period of about 1 minute. Optionally step g further comprises agitating the substrate in the treatment solution.

Optionally the treatment solution is applied to the substrate using a brush.

Optionally the color of the stained substrate and the treated substrate is evaluated by measuring the L*a*b* values of the stained substrate and the treated substrate.

According to a further aspect of the invention there is provided a staining solution comprising instant coffee powder at a concentration of from about 0.10 g/ml to about 0.60 g/ml and from about 0.5% to about 2.0% by volume glacial acetic acid. Optionally the staining solution comprises instant coffee powder at a concentration of from about 0.20 g/ml to about 0.40 g/ml and from about 0.5% to about 2.0% by volume glacial acetic acid. Optionally the staining solution comprises instant coffee powder at a concentration of about 0.25 g/ml and about 1.25% by volume glacial acetic acid.

According to a further aspect of the invention there is also provided use of a staining solution as disclosed herein in the methods disclosed herein.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### DETAILED DESCRIPTION

The following description of the preferred embodiment(s) is merely exemplary in nature and is in no way intended to limit the invention, its application, or uses.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight. The amounts given are based on the active weight of the material.

The methods of the present invention provide a way of demonstrating instantly the whitening and/or stain removal efficacy of an oral care composition. These in vitro methods can therefore be used to demonstrate the benefits of an oral care composition to consumers in a compelling and engaging way.

The methods of the present invention utilize a staining solution to stain a substrate before the application of an oral care composition. The substrate is selected to represent the tooth enamel surface of a mammal. The staining solution comprises coffee. It has surprisingly been found that coffee solution can be used in the methods of the present invention to demonstrate stain removal by an oral care composition. For example, in the methods of the present invention, a staining solution comprising coffee can be used to demonstrate the stain removal and/or whitening efficacy of an oral care composition such as a dentifrice. In certain embodiments, the staining solution comprises instant coffee powder, for example freeze-dried or spray-dried coffee powder or granules. In certain embodiments the staining solution comprises an instant coffee-chicory mixture. In certain embodiments the staining solution comprises an instant coffee-chicory mixture comprising from about 50 to about 80% by weight coffee. In certain embodiments the staining solution comprises an instant coffee-chicory mixture comprising from about 60 to about 70% by weight coffee. In certain embodiments the staining solution comprises an instant coffee-chicory mixture comprising about 60% by weight coffee. In certain embodiments the staining solution comprises an instant coffee-chicory mixture comprising about 70% by weight coffee. In certain embodiments the staining solution comprises NESTLE NESCAFÉ SUNRISE instant coffee, NESTLE NESCAFÉ SUNRISE Premium instant coffee, NESTLE NESCAFÉ SUNRISE STRONG instant coffee and mixtures of one or more thereof. In the methods of the present invention, the staining solution comprising coffee is used to stain a substrate which represents a tooth surface. Thus the coffee stained substrate can be used to demonstrate the stain removal and/or whitening efficacy of an oral care composition. This stain removal and/or whitening can be demonstrated much more quickly using the methods of the invention than if the oral care composition was applied by a consumer to his or her own teeth.

In certain embodiments, the staining solution comprises instant coffee powder at a concentration of from about 0.10 g/ml to about 0.60 g/ml, for example from about 0.10 g/ml to about 0.50 g/ml, from about 0.10 g/ml to about 0.40 g/ml, from about 0.10 g/ml to about 0.30 g/ml, from about 0.20 g/ml to about 0.60 g/ml or from about 0.25 g/ml to about 0.60 g/ml. In certain embodiments the staining solution comprises about 50 g coffee powder in about 150 ml water.

In certain embodiments, the staining solution comprises both coffee powder and an acid. In certain embodiments, the staining solution comprises acetic acid. In certain embodiments, the staining solution comprises from about 0.5% to about 2.0% by volume glacial acetic acid. For example the staining solution may comprise from about 0.5% to about 1.5% or from about 1.0% to about 1.5% by volume glacial acetic acid. In certain embodiments the staining solution comprises about 1.25% by volume glacial acetic acid.

In certain embodiments, the staining solution comprises both instant coffee powder and glacial acetic acid. For example, in certain embodiments the staining solution comprises from about 0.10 g/ml to about 0.60 g/ml, from about 0.10 g/ml to about 0.50 g/ml, from about 0.10 g/ml to about 0.40 g/ml, from about 0.10 g/ml to about 0.30 g/ml, from about 0.20 g/ml to about 0.60 g/ml or from about 0.25 g/ml to about 0.60 g/ml instant coffee powder and from about 0.5% to about 2.0% by volume glacial acetic acid, from about 0.5% to about 1.5% or from about 1.0% to about 1.5% by volume glacial acetic acid. In certain embodiments the staining solution comprises about 50 g coffee powder in about 150 ml water together with about 50 ml 5% glacial acetic acid. The staining solutions of the present invention evenly stain the substrate and do not fade rapidly. For example, the staining solutions of the present invention stain the substrate for at least about 1 hour, at least about 2 hours, at least about 4 hours, at least about 8 hours, at least about 12 hours, at least about 24 hours or at least about 48 hours. Once dried on the substrate, the staining solutions of the present invention cannot be removed by merely rinsing the stained with water.

In certain embodiments, the substrate comprises solid calcium carbonate. In certain embodiments, the substrate comprises the exoskeleton of a marine bivalve mollusk, for example of the family Arcidae. In certain embodiments, the substrate is a shell, for example a white ark shell. In certain embodiments the substrate is a medium size white ark shell approximately 1-3 inches (2.54 to 7.62 cm) in width. In certain embodiments the substrate is of uniform white appearance with substantially no uneven spots or discoloration and substantially no damage to the surface. In the methods of the invention, the substrate acts as an in vitro model for the tooth enamel of a mammalian tooth surface, allowing the effect of oral care compositions in cleaning oral surfaces (such a tooth surfaces) to be demonstrated. In certain embodiments, white ark shells are washed in tap water prior to use in the methods of the invention in order to remove any trapped dirt.

In certain embodiments, the methods of the present invention are used to demonstrate the stain removal efficacy of a dentifrice. For example, the methods may be used to demonstrate, in vitro, the stain removal and/or whitening effects of a dentifrice. By comparing the substrate before and after application of a treatment solution comprising an oral care composition, the stain removal and/or whitening effects of an oral care composition can be demonstrated to a consumer. In the methods of the present invention, the stain removal and/or whitening effects of an oral care composition can be demonstrated in a few minutes or hours, rather than in the days or weeks required if a consumer were to use the oral care composition on their own teeth and then observe the effects.

In certain embodiments the oral care composition is a dentifrice comprising pyrophosphates, peroxide, SLS and/or high cleaning silica. In certain embodiments the oral care composition is a dentifrice comprising hydrogen peroxide. In certain embodiments the oral care composition is a dentifrice comprising sodium tripolyphosphate, tetra potassium pyrophosphate and high cleaning silica. In certain embodiments the oral care composition is a dentifrice comprising about 2-4 weight % sodium tripolyphosphate, about 2-4 weight % tetra potassium pyrophosphate and about 18 to about 26 weight % high cleaning silica. In certain embodiments the oral care composition is COLGATE VISIBLE WHITE, COLGATE OPTIC WHITE or COLGATE LUMINOUS WHITE toothpaste.

In the methods of the present invention, a treatment solution comprising the oral care composition is applied to the substrate to demonstrate the cleaning effectiveness of the oral care composition by the removal of the coffee staining from the substrate. In certain embodiments the treatment solution comprises a slurry of the oral care composition and water, for example a slurry of dentifrice and water. In certain embodiments, the treatment solution comprises a slurry of dentifrice and water in a ratio of from about 1:2 to about 1:4 by weight. For example, the treatment solution may comprise dentifrice:slurry in a ratio of from about 1:2.5 to about 1:3.5, or for example a ratio of about 1:3 by weight. The treatment solution may be prepared by thoroughly mixing a dentifrice and water in an appropriate ratio with a magnetic stirrer immediately prior to application to the substrate.

In certain embodiments, the substrate is washed and air-dried and is then immersed in the staining solution. By “air-dried” it is meant that the substrate is allowed to dry at room temperature without aid from any heating apparatus. Once “air dry”, the substrate is substantially dry to the touch and any surface water has substantially evaporated. In certain embodiments the substrate is immersed in the staining solution for from about 20 to about 60 minutes. For example, the substrate may be immersed in the staining solution for from about 20 to about 50 minutes, from about 25 to about 50 minutes, from about 30 to about 50 minutes or from about 40 to about 50 minutes. In certain embodiments the substrate is immersed in the staining solution for about 45 minutes. The substrate can then be removed from the staining solution and allowed to air dry at room temperature such that the substrate is substantially dry to the touch and such that substantially all surface water has evaporated.

In certain embodiments the stained substrate is mounted on a slide or flat stick to facilitate application of the treatment solution comprising the oral care composition.

The stained substrate is assessed for staining by the staining solution by evaluating the color of the stained substrate. The color of the stained substrate is then compared to the color of the substrate after application of the treatment solution for visual comparison. In certain embodiments, this assessment is done by the naked eye. For example, a consumer may be shown the stained substrate both before and after application of the treatment solution.

In certain embodiments the evaluation of the color of the stained substrate is quantified using measurement of the L*a*b* color space. (L*a*b* refers to stain score in accordance with the Commission International de L'Eclairage Laboratory (CIELAB) color scale. L* (lightness-darkness scale), a* (red-green chroma) and b* (yellow-blue chroma)).

From measurement of the L*a*b* values, a whitening index can be calculated: ΔW*=W*final−W*initial, where W*=(a*²+b*²+(L*−100)²)^{1/2}. L*a*b* values can be measure using an optic shade-taking system to analyse and identify the color of a substrate. For example, the color of the stained substrate can be measured using a Spectroshade machine.

Following evaluation of the color of the stained substrate, the treatment solution is applied to the substrate. In certain embodiments the treatment solution is applied to the substrate by immersing the substrate in the treatment solution. In certain embodiments the substrate may be immersed in the treatment solution and agitated gently. In certain embodiments the substrate may be immersed in the treatment solution by immersing the substrate in a beaker comprising the treatment solution. In certain embodiments the treatment solution is applied to the substrate using a brush, for example using a toothbrush. In certain embodiments, the treatment solution is applied to the substrate by hand using a manual or electric toothbrush.

In certain embodiments the substrate is immersed in the treatment solution for a period of from about 20 s to about 2 minutes. In certain embodiments the substrate is immersed in the treatment solution for a period of from about 30 s to about 100 s, from about 45 s to about 90 s, from about 45 s to about 90 s or from about 50 s to about 80 s. In certain embodiments the substrate is immersed in the treatment solution for a period of about 1 minute.

In certain embodiments the treatment solution is applied to the substrate by manually brushing the substrate with the treatment solution for a period of from about 30 s to about 100 s, from about 45 s to about 90 s, from about 45 s to about 90 s or from about 50 s to about 80 s. In certain embodiments the substrate is brushed with the treatment solution for a period of about 60 s.

Following application of the treatment solution to the substrate, the color of the treated substrate is evaluated. In certain embodiments, this assessment is done by the naked eye. In certain embodiments the evaluation of the color of the treated substrate is quantified using measurement of the L*a*b* color space. From measurement of the L*a*b* values, a whitening index can be calculated. Comparison of the color of the treated substrate with the color of the stained substrate allows the efficacy of the oral care composition to be demonstrated.

### EXAMPLE

### Example 1

A staining solution is prepared using 50 g of Nestle Classic Sunrise instant coffee dissolved in 150 ml tap water and 50 ml 5% glacial acetic acid. This is mixed well at 60° C. for about 5 minutes with continuous stirring. A dentifrice slurry treatment solution is prepared by mixing 1 part Colgate Visible White toothpaste (comprising 3.00% sodium tripolyphosphate, 2.44% tetra potassium pyrophosphate and 22.0% high cleaning silica) with 3 parts tap water and mixing thoroughly with a magnetic stirrer. 50 white Ark shells of size 1-3 inches (2.54-7.62 cm) are washed in tap water and placed in a container comprising the staining solution in a sufficient quantity for the shells to be submerged. The shells are soaked at room temperature for 45 minutes and are then removed and air-dried. Stained shells are stuck to one end of a flat stick and placed in a beaker containing 50-100 ml slurry or 50-100 ml tap water as a control. 25 shells are submerged in a treatment solution and 25 shells are submerged in water as a control. The shells are immersed in the treatment solution (or water) for 1 minute with mild agitation. The shells are then removed from the treatment solution or water, rinsed in tap water and air-dried.

Both the treated and control shells are evaluated for staining using a Spectroshade machine. The results for the treated sea shells are shown in Table 1:

TABLE 1

| Treated | | | | W* = (a*² + b*² + |
|---|---|---|---|---|
| Sea Shells | L* | a* | b* | (L* − 100)²)^{1/2} |
| 1 | 82.2 | −1.4 | 7 | 19.18 |
| 2 | 86.8 | −0.9 | 7.6 | 15.26 |
| 3 | 89.4 | −0.9 | 4.5 | 11.55 |
| 4 | 87 | −0.9 | 7.1 | 14.84 |
| 5 | 83 | 0.4 | 6.9 | 18.35 |
| 6 | 79 | −0.7 | 7.8 | 22.41 |
| 7 | 79.9 | −1.7 | 7.7 | 21.59 |
| 8 | 79 | 1 | 9.6 | 23.11 |
| 9 | 81.5 | −0.2 | 5.6 | 19.33 |
| 10 | 85.1 | −1.2 | 6.3 | 16.22 |
| 11 | 78.5 | −0.2 | 8.9 | 23.27 |
| 12 | 85.5 | −0.9 | 6.1 | 15.76 |
| 13 | 81.7 | 0.4 | 8.7 | 20.27 |
| 14 | 79.4 | −0.6 | 6.8 | 21.70 |
| 15 | 75.6 | 0.9 | 0.8 | 24.43 |
| 16 | 79.8 | 0.9 | 7.2 | 21.46 |
| 17 | 84.7 | −1.3 | 6.6 | 16.71 |
| 18 | 84.1 | −0.1 | 6.5 | 17.18 |
| 19 | 81.1 | 0.8 | 7.6 | 20.39 |
| 20 | 85.8 | −1 | 6.5 | 15.65 |
| 21 | 84.4 | 0.3 | 7.8 | 17.44 |
| 22 | 82.1 | −1.9 | 7.9 | 19.66 |
| 23 | 88.3 | −1.4 | 6.7 | 13.56 |
| 24 | 78.6 | −0.3 | 7.1 | 22.55 |
| 25 | 86.5 | −0.9 | 5.1 | 14.46 |
| Avg. | 82.76 | −0.472 | 6.816 | 18.65 |
| StdDev. | 3.55 | 0.86 | 1.68 | 3.46 |

The results for the untreated (stained) shells are shown in Table 2:

TABLE 2

| Stained Sea | | | | W* = (a*² + b*² + |
|---|---|---|---|---|
| Shells | L* | a* | b* | (L* − 100)²)^{1/2} |
| 1 | 67.4 | 2.1 | 33.5 | 46.79 |
| 2 | 57.9 | 3.1 | 26.6 | 49.90 |
| 3 | 64.7 | 3.1 | 23.2 | 42.35 |
| 4 | 73.1 | 3.1 | 29.3 | 39.90 |
| 5 | 70.8 | 3.4 | 28.9 | 41.22 |
| 6 | 64.7 | 3.1 | 23.2 | 42.35 |
| 7 | 61.6 | 2.5 | 22 | 44.33 |
| 8 | 64.7 | 3.1 | 23.2 | 42.35 |
| 9 | 65 | 4.5 | 26.3 | 44.01 |
| 10 | 74.6 | 3.5 | 29.5 | 39.09 |
| 11 | 61.2 | 4 | 20.4 | 44.02 |
| 12 | 69.9 | 4.1 | 26 | 39.99 |
| 13 | 70.8 | 4.1 | 28.8 | 41.22 |
| 14 | 62.1 | 4.6 | 23.9 | 45.04 |
| 15 | 72.2 | 4.2 | 28.6 | 40.11 |
| 16 | 69.6 | 3.9 | 27.9 | 41.45 |
| 17 | 71.8 | 3.2 | 27.6 | 39.59 |
| 18 | 67.6 | 3.1 | 23.2 | 39.97 |
| 19 | 56.5 | 5.7 | 22.6 | 49.35 |
| 20 | 76 | 3.8 | 29.8 | 38.45 |
| 21 | 64.8 | 5.8 | 27.2 | 44.86 |
| 22 | 65.3 | 4.8 | 27.1 | 44.29 |
| 23 | 69.8 | 4.3 | 27.1 | 40.80 |
| 24 | 71.1 | 4.5 | 28.6 | 40.91 |
| 25 | 64 | 4.8 | 24.8 | 43.98 |
| Avg. | 67.088 | 3.856 | 26.372 | 42.65 |
| StdDev. | 5.047881 | 0.91108 | 3.073749 | 3.0 |

The whitening index for both the treated and untreated (stained) shells was then calculated. The results are shown in Table 3:

TABLE 3

| | | | | | Whitening |
|---|---|---|---|---|---|
| | | L* | a* | b* | Index |
| Stained | Avg. | 67.088 | 3.856 | 26.372 | 42.652339 |
| Shells | | | | | |
| Treated | Avg. | 82.76 | −0.472 | 6.816 | 18.653035 |
| Shells | | | | | |

A clear difference in the whitening index of the treated and untreated (stained) shells is seen, demonstrating that the efficacy of the oral care composition Colgate Visible White can be demonstrated quickly and easily to consumers. The effects are also visible to the naked eye.

As those skilled in the art will appreciate, numerous changes and modifications may be made to the embodiments described herein without departing from the spirit of the invention. It is intended that all such variations fall within the scope of the appended claims.

1. An in vitro method for demonstrating the stain removal efficacy of an oral care composition comprising
a. providing a substrate,
b. providing a staining solution,
c. providing a treatment solution comprising the oral care composition,
d. immersing the substrate in the staining solution,
e. removing the substrate from the staining solution,
f. evaluating the color of the stained substrate,
g. applying the treatment solution to the substrate and
h. evaluating the color of the treated substrate,
wherein the staining solution comprises coffee.

2. The method according to claim 1 wherein the staining solution comprises instant coffee powder.

3. The method according to claim 1 wherein the staining solution comprises instant coffee powder and an acid.

4. The method according to claim 1 wherein the staining solution comprises instant coffee powder at a concentration of from about 0.10 g/ml to about 0.60 g/ml.

5. The method according to claim 1 wherein the staining solution comprises instant coffee powder at a concentration of from about 0.20 g/ml to about 0.40 g/ml.

6. The method according to claim 1 wherein the staining solution comprises instant coffee powder at a concentration of about 0.25 g/ml.

7. The method according to claim 1 wherein the staining solution comprises acetic acid.

8. The method according to claim 1 wherein the staining solution comprises from about 0.5% to about 2.0% by volume glacial acetic acid.

9. The method according to claim 1 wherein the staining solution comprises about 1.25% by volume glacial acetic acid.

10. The method according to claim 1 wherein the staining solution comprises instant coffee powder at a concentration of about 0.25 g/ml and glacial acetic acid at a concentration of about 1.25% by volume.

11. The method according to claim 1 wherein the substrate comprises calcium carbonate.

12. The method according to claim 1 wherein the substrate comprises the exoskeleton of a marine bivalve mollusk.

13. The method according to claim 1 wherein the substrate comprises the exoskeleton of a marine bivalve mollusk in the family Arcidae.

14. The method according to claim 1 wherein the oral care composition is a dentifrice.

15. The method according to claim 1 wherein the oral care composition comprises silica and pyrophosphate.

16. The method according to claim 1 wherein the treatment solution comprises a slurry of an oral care composition and water.

17. The method according to claim 1 wherein the treatment solution comprises a slurry of dentifrice and water in a ratio of from about 1:2 to about 1:4 by weight.

18. The method according to claim 1 wherein the treatment solution comprises a slurry of dentifrice and water in a ratio of about 1:3 by weight.

19. The method according to claim 1 wherein step d comprises immersing the substrate in the staining solution for from about 20 to about 60 minutes.

20. The method according to claim 1 wherein step d comprises immersing the substrate in the staining solution for about 45 minutes.

21. The method according to claim 1 additionally comprising step e (ii) wherein the substrate is air-dried.

22. The method according to claim 1 wherein step g comprises immersing the substrate in the treatment solution for a period of from about 20 seconds to about 2 minutes.

23. The method according to claim 1 wherein step g comprises immersing the substrate in the treatment solution for a period of about 1 minute.

24. The method according to claim 22 wherein step g further comprises agitating the substrate in the treatment solution.

25. The method according to claim 1 wherein the treatment solution is applied to the substrate using a brush.

26. The method according to claim 1 wherein the color of the stained substrate and the color of the treated substrate is evaluated by measuring the L*a*b* values of the stained substrate and the treated substrate.

27. A staining solution comprising instant coffee powder at a concentration of from about 0.10 g/ml to about 0.60 g/ml and from about 0.5% to about 2.0% by volume glacial acetic acid.

28. The staining solution of claim 27 comprising instant coffee powder at a concentration of from about 0.20 g/ml to about 0.40 g/ml and from about 0.5% to about 2.0% by volume glacial acetic acid.

29. The staining solution of claim 27 comprising instant coffee powder at a concentration of about 0.25 g/ml and about 1.25% by volume glacial acetic acid.

30. (canceled)
